(19) United States
(12) Patent Application Publication
(10) Pub. No.: US 2022/0211960 A1
(43) Pub. Date: Jul. 7, 2022

(54) A SYSTEM FOR DELIVERING INHALED THERAPIES

(71) Applicant: RESPINOVA LTD., Herzliya Pituach (IL)

(72) Inventor: Yuval AVNI, Tel Aviv (IL)

(21) Appl. No.: 17/609,503

(22) PCT Filed: May 7, 2020
(86) PCT No.: PCT/IL2020/050500

§ 371 Date: Nov. 8, 2021

Related U.S. Application Data
(60) Provisional application No. 62/844,741, filed on May 8, 2019.

(51) Int. Cl. A61M 16/00; A61M 11/06; A61M 16/10; A61M 16/12; A61M 16/20; A61M 16/16

(52) U.S. Cl. CPC A61M 16/024; A61M 2230/432; A61M 16/1005; A61M 16/1085; A61M 16/125; A61M 16/204; A61M 16/205; A61M 16/0066; A61M 16/161; A61M 2016/0027; A61M 2016/0039; A61M 2205/14; A61M 2205/3358; A61M 2205/3368; A61M 2205/3375; A61M 11/06

(57) ABSTRACT

An inhaler comprises (a) a first passage having a first open end and a second open end; (b) a pressure source configured for generating an airflow; said pressure source connected to said first open end of said first passage; (c) a rotating shutter configured for blocking and releasing said airflow; (d) a mouthpiece being in fluid communication with second open end of said first passage; said mouthpiece configured for delivering a modulated airflow to a patient's airway. The inhaler further comprises a second passage having first open end and a second open end; said first open end of said second passage is in fluid communication with said mouthpiece; said second open end of said second passage is vented to ambient air; said first and second passages are arranged such that said rotating shutter blocks and releases said first and second passages in an alternate manner.

### FIELD OF THE INVENTION

The present invention relates to medical devices for preventing and treating respiratory diseases and, more specifically, to inhalers provided with inlet and outlet passages blockable and releasable in an alternate manner.

### BACKGROUND OF THE INVENTION

WO2015186124 discloses a pulsating therapeutic inhaler generating pneumatic pulses for treating respiratory disorders. The aforesaid inhaler comprises: (a) a linear passage having an elongate axis; the linear passage configured to conduct a fluid flow in a laminar manner; (b) a patient interface fluidly connectable to the patient's respiratory tract having an aperture fluidly connectable to the passage; and (c) a shutter disposed between the passage and the aperture configured to modulate a fluid pressure within the fluid flow; the shutter comprising a disc having at least one cutout and rotating about an axis parallel to the passage axis. The cutout has four cornered perimeter thereof with two side portions and two circumferential arcs configured relative to the rotation axis. The side portions are circumferentially anti-symmetrical relative to the aperture.

The pulsing inhalers know in the art have an air passage feeding an airflow into a patent's airway. During the inhalation phase air fed by the inhaler flows into the patient's airway. At the phase of exhalation a part of exhaled air regresses into the feeding air passage. The described effect is known of a dead-space effect. Thus, there is a long-felt need of providing an inhaler minimizing the aforesaid effect and discharging the exhaled air all-out.

### SUMMARY OF THE INVENTION

It is hence one object of the invention to disclose an inhaler comprising: (a) a first passage having a first open end and a second open end; (b) a pressure source configured for generating an airflow; said pressure source connected to said first open end of said first passage; (c) a rotating shutter configured for blocking and releasing said airflow; (d) a mouthpiece being in fluid communication with second open end of said first passage; said mouthpiece configured for delivering a modulated airflow to a patient's airway.

It is a core purpose of the invention to provide the inhaler comprising a second passage having first open end and a second open end. The first open end of said second passage is in fluid communication with said mouthpiece. The second open end of said second passage is vented to ambient air. The first and second passages are arranged such that said rotating shutter blocks and releases said first and second passages in an alternate manner.

Another object of the invention is to disclose the first and second passages arranged nearby in a parallel manner.

A further object of the invention is to disclose the first passage arranged within the second passage.

A further object of the invention is to disclose the first passage having a passage gate valve configured for controlling a flow rate within said first passage.

A further object of the invention is to disclose the first passage having a branch inlet configured for connecting a humidifier unit.

A further object of the invention is to disclose the nebulizer gate valve selected from the group consisting of a mechanically actuated valve, an electrically actuated valve, a pneumatically actuated valve, a magnetically actuated valve and any combination thereof.

A further object of the invention is to disclose the nebulizer branch inlet comprising a cylinder reciprocatively displaceable within said nebulizer branch inlet between an open position at an inhalation phase and a closed position at an exhalation phase and. In said open position, said nebulizer is fluidly connected with said mouthpiece; said closed position blocks fluid communication between said nebulizer and said mouthpiece.

A further object of the invention is to disclose the nebulizer which is connectable to an air pressure source.

A further object of the invention is to disclose a connection of said air pressure source to said nebulizer comprising an air pressure source gate valve.

A further object of the invention is to disclose the nebulizer connectable to an oxygen source.

A further object of the invention is to disclose the inhaler comprising a control unit configured for controlling an element selected from the group consisting of said shutter, said passage gate valve, said humidifier gate valve, air pressure source gate valve and any combination thereof; said control unit is preprogrammed for implementing a predetermined treatment protocol.

A further object of the invention is to disclose the inhaler comprising a pressure sensor providing a feedback to said control unit during implementing said predetermined treatment protocol.

A further object of the invention is to disclose the inhaler comprising a control unit configured for controlling an element selected from the group consisting of said shutter, said passage gate valve, said humidifier gate valve, air pressure source gate valve, said airflow heater and any combination thereof; said control unit is preprogrammed for implementing a predetermined treatment protocol.

A further object of the invention is to disclose the inhaler comprising a sensor selected from the group consisting of a pressure sensor located at said first open end of said first passage and configured for detecting air pressure provided by said pressure source, a flow rate sensor located at said first open end of said first passage and configured to detect an airflow rate provided by said pressure sensor, a presence sensor located at said humidifier branch inlet and configured for detecting presence of said humidifier, a humidity sensor located at said humidifier branch inlet and configured for detecting air humidity provided by said humidifier, a barometric sensor configured to detect barometric pressure of ambient air, a an oxygen sensor located at said oxygen source, a presence sensor located at said nebulizer branch inlet and configured for detecting presence of said nebulizer, a pressure sensor located at said mouthpiece and configured for detecting air pressure within said mouthpiece, a lung gases sensor configured for detecting a gas composition of exhaled air, a humidity sensor located at said mouthpiece and configured for detecting air humidity within said mouthpiece, a temperature sensor located at said mouthpiece and configured for detecting temperature within said mouthpiece, an acoustic sensor located at said mouthpiece and configured for detecting breathing sounds within said patient's airway, an acoustic sensor located at a patient's body and configured for detecting breathing sounds within said patient's airway and any combination thereof.

A further object of the invention is to disclose a method of preventing and treating respiratory diseases; said method comprising steps of: (a) providing an inhaler further comprising (i) a first passage having a first open end and a second open end; (ii) a pressure source configured for generating an airflow; said pressure source connected to said first open end of said first passage; (iii) a rotating shutter configured for blocking and releasing said airflow; (iv) a mouthpiece being in fluid communication with second open end of said first passage; said mouthpiece configured for delivering a modulated airflow to a patient's airway; (v) said inhaler comprises a second passage having first open end and a second open end; said first open end of said second passage is in fluid communication with said mouthpiece; said second open end of said second passage is vented to ambient air; said first and second passages are arranged such that said rotating shutter blocks and releases said first and second passages in an alternate manner; (b) providing pneumatic pulses to said mouthpiece by means of blocking and releasing said airflow within said first passage; (c) venting said mouthpiece to ambient air. Steps b and c are executed in an alternate manner.

A further object of the invention is to disclose the step of providing pneumatic pulses comprising controlling a flow rate within said first passage by a passage gate valve configured for.

A further object of the invention is to disclose the step of providing pneumatic pulses comprising humidifying said airflow within said first passage by a humidifier connected to a branch inlet.

A further object of the invention is to disclose the step of humidifying said airflow comprising controlling a flow rate between said humidifier unit and said first passage by a humidifier gate valve.

A further object of the invention is to disclose the sub-step of humidifying said airflow comprising controlling a flow rate between said humidifier unit and said first passage by a humidifier gate valve.

A further object of the invention is to disclose the step of providing pneumatic pulses comprising a sub-step of nebulizing a medicament into said second passage.

A further object of the invention is to disclose the sub-step of nebulizing said medicament comprising positioning a reciprocatively displaceable cylinder within said nebulizer branch inlet into an open position at an inhalation phase and into a closed position at an exhalation phase such that said nebulizer is fluidly connected with said mouthpiece in said open position and a fluid communication between said nebulizer and said mouthpiece is blocked in said closed position.

A further object of the invention is to disclose the method comprising a step of a control unit configured for controlling an element selected from the group consisting of said electric motor, said passage gate valve, said humidifier gate valve and any combination thereof; said control unit is preprogrammed for implementing a predetermined treatment protocol.

A further object of the invention is to disclose the inhaler comprising a step of controlling said element feedback selected from the group consisting of said electric motor, said passage gate valve, said humidifier gate valve and any combination thereof comprises getting feedback from a pressure sensor.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic diagram of an inhaler provided with inlet and outlet passages blockable and releasable in an alternate manner;

FIG. 2 is a schematic diagram of an inhaler provided with a nebulizer;

FIGS. 3a to 3d illustrate operation a gate valve;

FIG. 4 is a schematic diagram of a sensor arrangement in an inhaler;

FIG. 5 is a schematic diagram of an internal heater within an air passage;

FIGS. 6a to 6c are schematic diagrams of a pipe arrangement in a mouthpiece of an inhaler; and

FIGS. 7a to 7d are schematic diagrams of alternative embodiments of a nebulizer gate valve in closed and open positions.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide an inhaler for preventing and treating respiratory diseases and a method of doing the same.

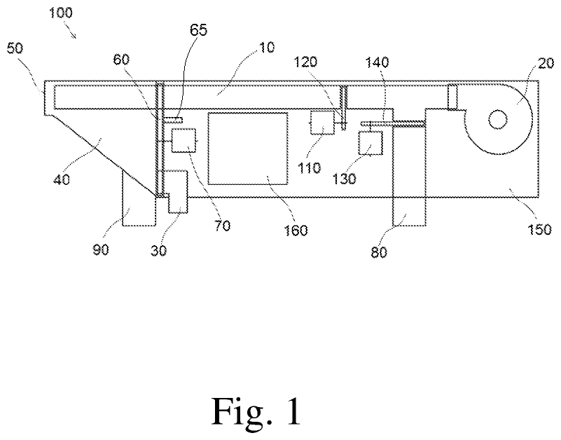

Reference is now made to FIG. 1 presenting a schematic diagram of inhaler 100 having housing 150 accommodating all its components. Specifically, an air flow from blower 20 is conducted by passage 10 which is in fluid communication with mouthpiece 50. A flow rate of the airflow is controlled by motorized gate valve 120 driven by actuator 110. Passage 10 is provided with humidifier branch inlet 80 allowing fluid communication between a humidifier (nor shown) connectable to humidifier branch inlet 80. Second passage 40 is provided with nozzle 30. In addition, second passage 40 is in fluid communication with nebulizer branch inlet 90 configured for connecting the humidifier thereto. Electric motor 70 drives rotating disc 60 having a cutout (not shown) such that first and second passages 10 and 40, respectively, are arranged such that rotating disc 60 blocks and releases first and second passages 10 and 40 in an alternate manner. It should be noticed that an excess pressure created by blower 20 when the cutout (not shown) on rotating disc 60 coincides with first passage 10 is vented to surrounding atmosphere when the cutout coincides with second passage 40. The exhaled air accommodated only in mouthpiece 50 is potentially pressurized back into the patient's airway. In other words, the dead-space effect is minimized.

Control unit 160 is configured for controlling electric motor 60, passage gate valve 120, humidifier gate valve 140 and air pressure source gate valve 270. Control unit 160 is preprogrammed for implementing a predetermined treatment protocol. Pressure sensor 65 provides a feedback to control unit 160 during implementing the predetermined treatment protocol.

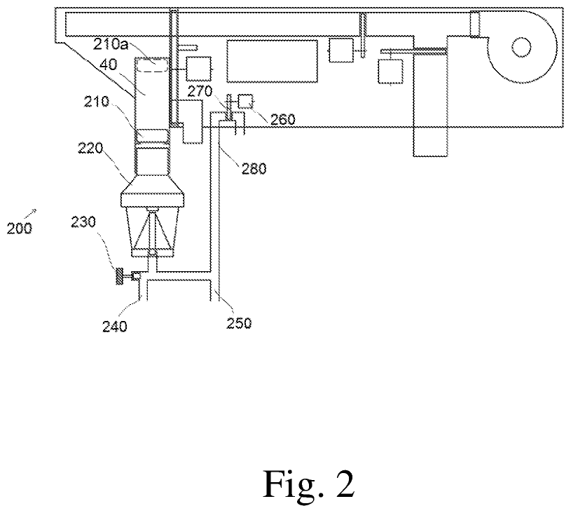

Reference is now made to FIG. 2 presenting arrangement 200 for connecting a nebulizer 220 to the inhaler 100 (not shown). The aforesaid nebulizer is connected to nebulizer branch inlet 40 having a cylinder reciprocatively displaceable within nebulizer branch inlet 40 between closed position 210 at an exhalation phase and open position 210a at an inhalation phase. In open position 210a, nebulizer 220 is fluidly connected with mouthpiece 50. Pipes 240 and 250 are connectable to an oxygen source and a pressurized air, respectively. An oxygen flow rate is controlled by tap 230. Gate valve 270 driven by actuator 260 is closable at inhalation phase and openable at exhalation phase. Numeral 245 refers to an oxygen sensor detecting an oxygen gas flow via pipe 240.

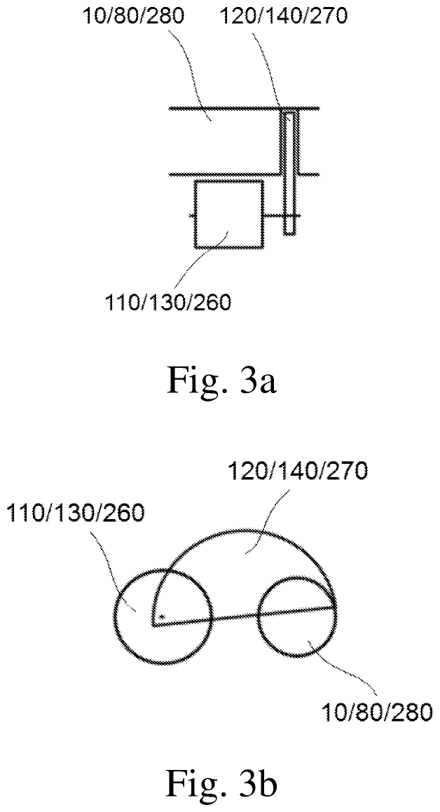

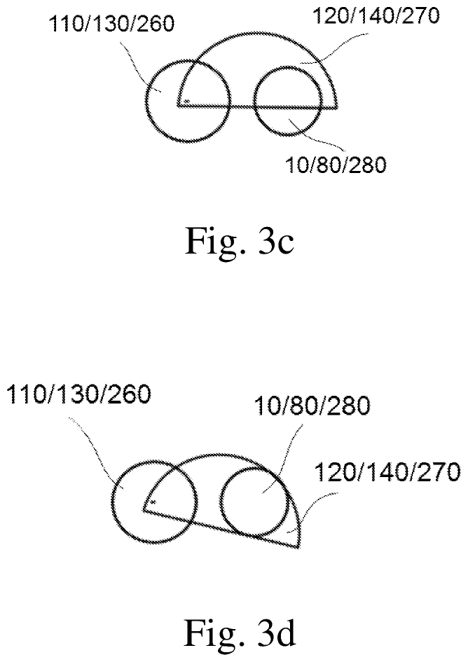

Reference is now made to FIGS. 3a to 3d illustrating operation of gate valves. As shown in FIG. 3a, the aforesaid gate valves are shown as shutters 120, 140 and 270. Shutter 120/140/270 is configured for opening and blocking passages 10/80/280. Shutter 120/140/270 is driven by actuators 110/130/260. FIGS. 3b to 3d, successive positions of shutter 120/140/270 which gradually blocks passage 10/80/280.

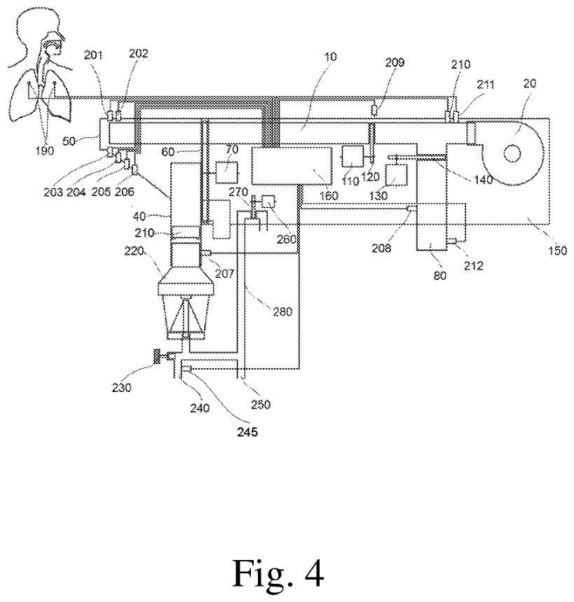

Reference is now made to FIG. 4 presenting a schematic diagram of a sensor arrangement in an inhaler. The inhaler can include at least one of the sensors provided below.

Airflow from blower 20 within passage 10 is characterized by air pressure detected by pressure sensor 210 and flow rate detected by flow rate sensor 211. Numeral 209 refers to a barometric sensor configured for measuring pressure of ambient air. The obtained value of barometric pressure is used by control unit in calculation of air pressure provided into mouthpiece 50.

Mouthpiece 50 can be provided with air pressure sensor 203 and flow rate sensor 202 detecting local air pressure and flow rate, respectively. Local relative humidity within mouthpiece 50 can be obtained by means of humidity and temperature sensors 205 and 206, respectively.

Acoustic sensor 201 located within mouthpiece 50 is designed for obtaining an acoustic pattern of inhalation/exhalation phases via the air flow. Contrary to this, acoustic sensor 190 can be placed on a patient's chest. The acoustic pattern obtained by sensors 201 and/or 190 can be used for adaptation of a therapeutic protocol to a specific patient's condition. Numeral 204 refers to a sensor which detects gas composition of air exhaled by the patient.

Presence of nebulizer 220 is reported by nebulizer present sensor 207. Presence of humidifier is reported by humidifier presence sensor 212. Humidity of the air flow in humidifier branch inlet is detected by humidity sensor 208.

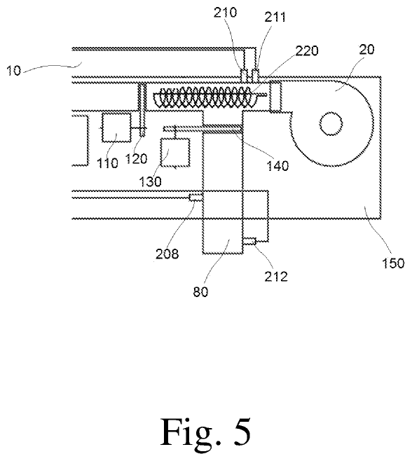

Reference is now made to FIG. 5 presenting heater 220 mounted within passage 10. An airflow conducted by passage 10 from blower 20 to mouthpiece (not shown) can be heated, as needed.

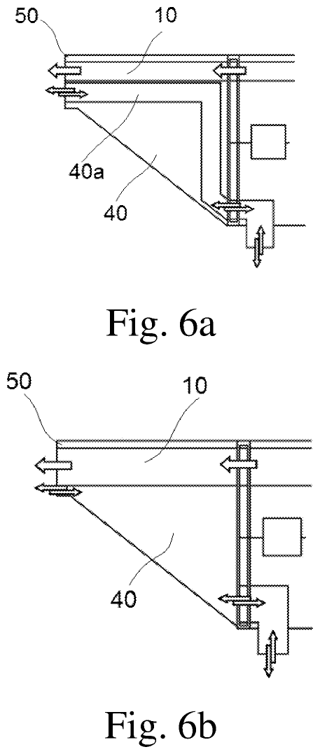

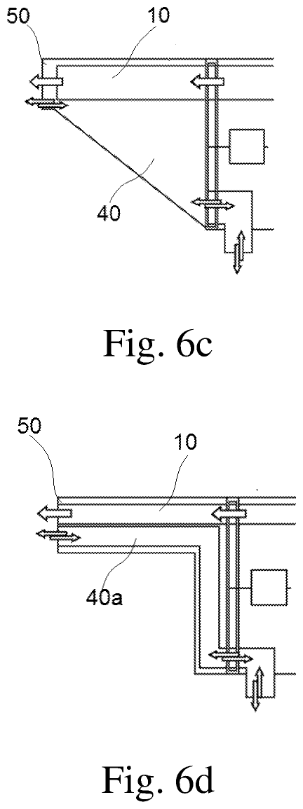

Reference is now made to FIGS. 6a to 6d presenting alternative embodiments of the present invention in terms of the minimal-dead-space conception. It should be mentioned that that in passage 10 there is air pressure directed to mouthpiece 50 while passage 40 (40a) provide a fluid communication with ambient atmosphere.

Specifically, FIG. 6a shows inhalation passage 10 and exhalation passage 40a. No distance between terminals of passages 10 and 40a and mouthpiece 50. Exhaled air is vented into ambient atmosphere. In other words, there is no dead space in the embodiment. Similar to FIG. 6a, in FIG. 6b, passage defined outer shell 40 serves an exhalation passage.

In FIG. 6c, inhalation passage 10 is displaced inwardly passage (shell) 40. Air within a space between edges of inhalation passage 10 and mouthpiece 50 after the exhalation phase is inhaled again.

An additional technical feature discriminating the embodiment of FIG. 6a from two others is in arranging two passages 10 and 40a within mouthpiece 50 nearby in a parallel manner. Contrary to this, FIGS. 6b and 6c present an arrangement where inhalation passage 10 is disposed in exhalation passage 40.

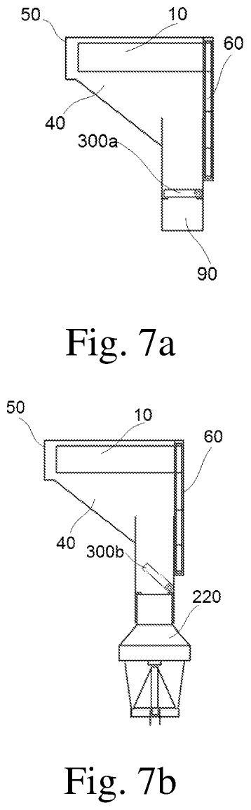

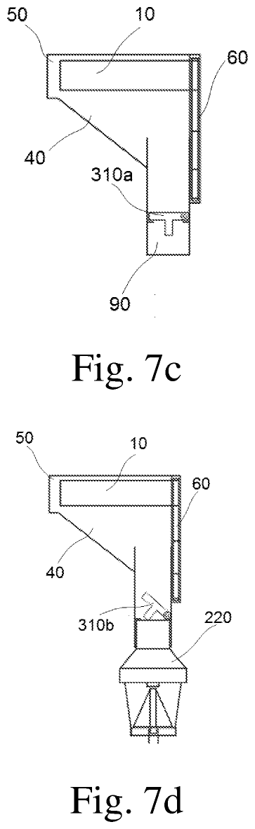

Reference is now made to FIGS. 7a to 7d presenting schematic diagrams of alternative embodiments of a nebulizer gate valve in closed and open positions. Specifically, FIGS. 7a and 7b show a pneumatically openable valve. Numerals 300a and 300b refer to closed and open positions, respectively. A mechanically actuated valve in closed and open positions 310a and 310b is shown in FIGS. 7c and 7d, respectively.

According to the present invention an inhaler is disclosed. The inhaler comprise: (a) a first passage having a first open end and a second open end; (b) a pressure source configured for generating an airflow; said pressure source connected to said first open end of said first passage; (c) a rotating shutter configured for blocking and releasing said airflow; (d) a mouthpiece being in fluid communication with second open end of said first passage; said mouthpiece configured for delivering a modulated airflow to a patient's airway.

It is a core feature of the invention to provide the inhaler comprising a second passage having first open end and a second open end. The first open end of said second passage is in fluid communication with said mouthpiece. The second open end of said second passage is vented to ambient air. The first and second passages are arranged such that said rotating shutter blocks and releases said first and second passages in an alternate manner.

According to one embodiment of the present invention, the first and second passages are arranged nearby in a parallel manner.

According to one embodiment of the present invention, the first passage is arranged within the second passage.

According to one embodiment of the present invention, the first passage has a passage gate valve configured for controlling a flow rate within said first passage.

According to another embodiment of the present invention, the first passage has a branch inlet configured for connecting a humidifier unit.

According to a further embodiment of the present invention, the nebulizer gate valve is selected from the group consisting of a mechanically actuated valve, an electrically actuated valve, a pneumatically actuated valve, a magnetically actuated valve and any combination thereof.

According to a further embodiment of the present invention, the nebulizer branch inlet comprises a cylinder reciprocatively displaceable within said nebulizer branch inlet between an open position at an inhalation phase and a closed position at an exhalation phase and. In said open position, said nebulizer is fluidly connected with said mouthpiece; said closed position blocks fluid communication between said nebulizer and said mouthpiece.

According to a further embodiment of the present invention, the nebulizer is connectable to an air pressure source.

According to a further embodiment of the present invention, a connection of said air pressure source to said nebulizer comprises an air pressure source gate valve.

According to a further embodiment of the present invention, the nebulizer is connectable to an oxygen source.

According to a further embodiment of the present invention, the inhaler comprises a control unit configured for controlling an element selected from the group consisting of said shutter, said passage gate valve, said humidifier gate valve, air pressure source gate valve, said airflow heater and any combination thereof; said control unit is preprogrammed for implementing a predetermined treatment protocol.

According to a further embodiment of the present invention, the inhaler comprises a sensor selected from the group consisting of a pressure sensor located at said first open end of said first passage and configured for detecting air pressure provided by said pressure source, a flow rate sensor located at said first open end of said first passage and configured to detect an airflow rate provided by said pressure sensor, a presence sensor located at said humidifier branch inlet and configured for detecting presence of said humidifier, a humidity sensor located at said humidifier branch inlet and configured for detecting air humidity provided by said humidifier, a barometric sensor configured to detect barometric pressure of ambient air, a an oxygen sensor located at said oxygen source, a presence sensor located at said nebulizer branch inlet and configured for detecting presence of said nebulizer, a pressure sensor located at said mouthpiece and configured for detecting air pressure within said mouthpiece, a lung gases sensor configured for detecting a gas composition of exhaled air, a humidity sensor located at said mouthpiece and configured for detecting air humidity within said mouthpiece, a temperature sensor located at said mouthpiece and configured for detecting temperature within said mouthpiece, an acoustic sensor located at said mouthpiece and configured for detecting breathing sounds within said patient's airway, an acoustic sensor located at a patient's body and configured for detecting breathing sounds within said patient's airway and any combination thereof.

According to a further embodiment of the present invention, a method of preventing and treating respiratory diseases is disclosed. The aforesaid method comprises steps of: (a) providing an inhaler further comprising (i) a first passage having a first open end and a second open end; (ii) a pressure source configured for generating an airflow; said pressure source connected to said first open end of said first passage; (iii) a rotating shutter configured for blocking and releasing said airflow; (iv) a mouthpiece being in fluid communication with second open end of said first passage; said mouthpiece configured for delivering a modulated airflow to a patient's airway; (v) said inhaler comprises a second passage having first open end and a second open end; said first open end of said second passage is in fluid communication with said mouthpiece; said second open end of said second passage is vented to ambient air; said first and second passages are arranged such that said rotating shutter blocks and releases said first and second passages in an alternate manner; (b) providing pneumatic pulses to said mouthpiece by means of blocking and releasing said airflow within said first passage; (c) venting said mouthpiece to ambient air. Steps b and c are executed in an alternate manner.

According to a further embodiment of the present invention, the step of providing pneumatic pulses comprises controlling a flow rate within said first passage by a passage gate valve configured for.

According to a further embodiment of the present invention, the step of providing pneumatic pulses comprises humidifying said airflow within said first passage by a humidifier connected to a branch inlet.

According to a further embodiment of the present invention, the step of humidifying said airflow comprises controlling a flow rate between said humidifier unit and said first passage by a humidifier gate valve.

According to a further embodiment of the present invention, the sub-step of humidifying said airflow comprises controlling a flow rate between said humidifier unit and said first passage by a humidifier gate valve.

According to a further embodiment of the present invention, the step of providing pneumatic pulses comprises a sub-step of nebulizing a medicament into said second passage.

According to a further embodiment of the present invention, the sub-step of nebulizing said medicament comprises positioning a reciprocatively displaceable cylinder within said nebulizer branch inlet into an open position at an inhalation phase and into a closed position at an exhalation phase such that said nebulizer is fluidly connected with said mouthpiece in said open position and a fluid communication between said nebulizer and said mouthpiece is blocked in said closed position.

According to a further embodiment of the present invention, the method comprises a step of a control unit configured for controlling an element selected from the group consisting of said electric motor, said passage gate valve, said humidifier gate valve and any combination thereof; said control unit is preprogrammed for implementing a predetermined treatment protocol.

According to a further embodiment of the present invention, the inhaler comprises a step of controlling said element feedback selected from the group consisting of said electric motor, said passage gate valve, said humidifier gate valve and any combination thereof comprises getting feedback from a pressure sensor.

1.-31. (canceled)

32. An inhaler comprising:
a. a first passage having a first open end and a second open end;
b. a pressure source configured for generating an airflow to be inhaled; said pressure source connected to said first open end of said first passage;
c. a second passage for conducting an exhaled airflow; said second passage having first open end and a second open end; said first open end of said second passage is in fluid communication with said mouthpiece; said second open end of said second passage is vented to ambient air;
d. a rotating shutter having at least one cutout; said rotating shutter configured for blocking and releasing said airflow;
e. a mouthpiece being in fluid communication with second open ends of said first and second passages; said mouthpiece configured for delivering said airflow to be inhaled to a patient's airway and exhausting said exhaled airflow therefrom;
wherein said first and second passages are spaced apart from each other are arranged such that said rotating shutter releases said to be inhaled airflow and said exhaled airflow conducted by said first and second passages, respectively, when said at least one cutout at least partially coincides with apertures of said first and second passages.

33. The inhaler according to claim 32, wherein at least one of the following is true:
a. said pressure source is selected from the group consisting of an air blower, a compressed-air flask, a compressed air line and any combination thereof;
b. said first passage has a passage gate valve configured for controlling a flow rate within said first passage;
c. said first passage has a humidifier branch inlet configured for connecting a humidifier unit;
d. said rotating shutter is a rotating disc having a cutout and blocking and releasing said first and second passages in an alternate manner; and
e. said rotating shutter is a rotating disc having a cutout and blocking and releasing said first and second passages in a synchronic manner.

34. The inhaler according to claim 33, wherein said humidifier branch pipe has a humidifier gate valve configured for controlling a flow rate between said humidifier unit and said first passage.

35. The inhaler according to claim 33, wherein said rotating disc is rotatably driven by an electric motor.

36. The inhaler according to claim 32, wherein at least one of the following is true:
a. said second passage has a nebulizer branch inlet configured for connecting a nebulizer;
b. said nebulizer branch inlet comprises a nebulizer gate valve configured for controlling a flow rate via said nebulizer branch inlet;
c. said nebulizer gate valve is selected from the group consisting of a mechanically actuated valve, an electrically actuated valve, a pneumatically actuated valve, a magnetically actuated valve and any combination thereof.

37. The inhaler according to claim 36, wherein said nebulizer gate valve comprises a cylinder reciprocatively displaceable within said nebulizer branch inlet between an open position at an inhalation phase and a closed position at an exhalation phase and; in said open position, said nebulizer is fluidly connected with said mouthpiece; said closed position blocks fluid communication between said nebulizer and said mouthpiece.

38. The inhaler according to claim 32, wherein said nebulizer is connectable to an air pressure source.

39. The inhaler according to claim 32, wherein a connection of said air pressure source to said nebulizer comprises an air pressure source gate valve closable at inhalation phase and openable at exhalation phase.

40. The inhaler according to claim 32, wherein said nebulizer is connectable to an oxygen source.

41. The inhaler according to claim 32, wherein said first passage comprises an airflow heater configured for heating said airflow within said first passage.

42. The inhaler according to claim 32 comprising a control unit configured for controlling an element selected from the group consisting of said shutter, said passage gate valve, said humidifier gate valve, air pressure source gate valve, said airflow heater and any combination thereof; said control unit is preprogrammed for implementing a predetermined treatment protocol.

43. The inhaler according to claim 42 comprising a sensor selected from the group consisting of a pressure sensor located at said first open end of said first passage and configured for detecting air pressure provided by said pressure source, a flow rate sensor located at said first open end of said first passage and configured to detect an airflow rate provided by said pressure sensor, a presence sensor located at said humidifier branch inlet and configured for detecting presence of said humidifier, a humidity sensor located at said humidifier branch inlet and configured for detecting air humidity provided by said humidifier, a barometric sensor configured to detect barometric pressure of ambient air, a an oxygen sensor located at said oxygen source, a presence sensor located at said nebulizer branch inlet and configured for detecting presence of said nebulizer, a pressure sensor located at said mouthpiece and configured for detecting air pressure within said mouthpiece, a lung gases sensor configured for detecting a gas composition of exhaled air, a humidity sensor located at said mouthpiece and configured for detecting air humidity within said mouthpiece, a temperature sensor located at said mouthpiece and configured for detecting temperature within said mouthpiece, an acoustic sensor located at said mouthpiece and configured for detecting breathing sounds within said patient's airway, an acoustic sensor located at a patient's body and configured for detecting breathing sounds within said patient's airway and any combination thereof.

44. The inhaler according to claim 43, wherein any sensor of the group provides a feedback to said control unit during implementing said predetermined treatment protocol.

45. A method of preventing and treating respiratory diseases; said method comprising steps of:
a. providing an inhaler further comprising
i. a first passage having a first open end and a second open end;
ii. a pressure source configured for generating an airflow; said pressure source connected to said first open end of said first passage;
iii. a second passage having first open end and a second open end; said first open end of said second passage is in fluid communication with said mouthpiece; said second open end of said second passage is vented to ambient air;
said inhaler comprises a second passage for conducting an exhaled airflow; said second passage having first open end and a second open end; said first open end of said second passage is in fluid communication with said mouthpiece; said second open end of said second passage is vented to ambient air;
said first and second passages are spaced apart from each other such that said rotating shutter releases said airflow to be inhaled and said exhaled airflow conducted by said first and second passages, respectively, when said at least one cutout at least partially coincides with apertures of said first and second passages.
b. providing pneumatic pulses to said mouthpiece by means of blocking and releasing said airflow within said first passage;
c. venting said mouthpiece to ambient air;
said steps b and c are executed in an alternate manner.

46. The method according to claim 45, wherein at least one of the following is true:
a. said step of providing pneumatic pulses comprises controlling a flow rate within said first passage by a passage gate valve configured for;
b. said step of providing pneumatic pulses comprises a sub-step of humidifying said airflow within said first passage by a humidifier connected to a branch inlet;
c. said step of providing pneumatic pulses comprises a sub-step of nebulizing a medicament into said second passage;
d. said method comprises a step of a control unit configured for controlling an element selected from the group consisting of said electric motor, said passage gate valve, said humidifier gate valve and any combination thereof; said control unit is preprogrammed for implementing a predetermined treatment protocol;

47. The method according to claim 46, wherein said sub-step of humidifying said airflow comprises controlling a flow rate between said humidifier unit and said first passage by a humidifier gate valve.

48. The inhaler according to claim 46, wherein said sub-step of nebulizing said medicament comprises positioning a reciprocatively displaceable cylinder within said nebulizer branch inlet into an open position at an inhalation phase and into a closed position at an exhalation phase such that said nebulizer is fluidly connected with said mouthpiece in said open position and a fluid communication between said nebulizer and said mouthpiece is blocked in said closed position.

49. The inhaler according to claim 46 comprising a step of controlling said element feedback selected from the group consisting of said electric motor, said passage gate valve, said humidifier gate valve, said airflow heater and any combination thereof comprises getting feedback from a pressure sensor.

50. The method according to claim 46 comprising a step of providing a feedback to said control unit by a sensor selected from the group consisting of a pressure sensor located at said first open end of said first passage and configured for detecting air pressure provided by said pressure source, a flow rate sensor located at said first open end of said first passage and configured to detect an airflow rate provided by said pressure sensor, a presence sensor located at said humidifier branch inlet and configured for detecting presence of said humidifier, a humidity sensor located at said humidifier branch inlet and configured for detecting air humidity provided by said humidifier, a barometric sensor configured to detect barometric pressure of ambient air, a an oxygen sensor located at said oxygen source, a presence sensor located at said nebulizer branch inlet and configured for detecting presence of said nebulizer, a pressure sensor located at said mouthpiece and configured for detecting air pressure within said mouthpiece, a lung gases sensor configured for detecting a gas composition of exhaled air, a humidity sensor located at said mouthpiece and configured for detecting air humidity within said mouthpiece, a temperature sensor located at said mouthpiece and configured for detecting temperature within said mouthpiece, an acoustic sensor located at said mouthpiece and configured for detecting breathing sounds within said patient's airway, an acoustic sensor located at a patient's body and configured for detecting breathing sounds within said patient's airway and any combination thereof.

51. The method according to claim 19 comprising a step of providing feedback from any sensor of the group said control unit during implementing said predetermined treatment protocol.
